(19) United States
(12) Patent Application Publication
(10) Pub. No.: US 2009/0209832 A1
(43) Pub. Date: Aug. 20, 2009

(54) TUBE MONITORING SYSTEM

(75) Inventors: Liam McDaid, Londonderry (IE); Heather Sayers, County Tyrone (GB); Mary McSorley, County Londonderry (GB)

Correspondence Address: VAN DYKE, GARDNER, LINN & BURKHART, LLP, SUITE 207, 2851 CHARLEVOIX DRIVE, S.E., GRAND RAPIDS, MI, 49546, US

(73) Assignee: UNIVERSITY OF ULSTER, County Londonderry (GB)

(21) Appl. No.: 12/280,473

(22) PCT Filed: Feb. 22, 2007
(86) PCT No.: PCT/EP07/01529

§ 371 Date: Jan. 16, 2009

(30) Foreign Application Priority Data
Feb. 23, 2006 (GB) 0603637.0

(51) Int. Cl. A61B 5/07

(52) U.S. Cl. 600/302

(57) ABSTRACT

A system for monitoring the location of a tube, especially a feeding tube. The system comprises an antenna located at an outlet of the tube; a wireless transmitter coupled to the antenna; and a sensor co-located with the antenna and arranged to generate an output which indicates at least one sensed characteristic, for example, pH level, of the tube's environment. The system includes a remote receiver device for receiving wireless signals, the receiver device including means for determining the strength of said received wireless signal, and means for determining said sensor output from said received wireless signal.

### FIELD OF THE INVENTION

The present invention relates to tube monitoring systems. The invention relates particularly to the monitoring of enteral feeding tubes.

### BACKGROUND TO THE INVENTION

Enteral feeding is the introduction of food directly into the stomach (or rarely into the duodenum or jejunum) bypassing the normal mouth/oesophagus route. Enteral feeds are given to patients who are unable to eat food normally or whose body demands exceed their oral nutritional intake. They can also be used for the administration of medicines, diagnostic imaging and in patients with bowel obstruction and bleeding. Enteral feeding usually demands that a tube is passed into the patient's stomach via the nose (nasogastric), the mouth (orogastric) or via Precutaneous Endoscopic Gastrostomy (PEG). While nasogastric and orogastric tubes afford relatively easy access, PEG tube introduction is a surgical procedure. Checking the position of nasogastric and orogastric tubes following insertion presents huge challenges to both formal and informal carers. According to the CREST guidelines, tube position must be either radiologically checked or aspirate via the tube must be tested for acidity. Radiology (x-ray) checking for most patients is not an option as the frequency of x-rays needed (up to six per day for a period of days, weeks, months or even years) exceeds safe exposure levels. The most common check undertaken is the aspirate test where fluid is withdrawn via the tube and placed on pH paper, which indicates the presence or absence of stomach acid. While this test method is successful in most cases, the pH test is not always correct. Between December 2002 and December 2004 at least eleven patients died in the United Kingdom as a direct result of misplaced tubes. It would be desirable, therefore, to improve the safety of enteral feeding.

### SUMMARY OF THE INVENTION

Accordingly, a first aspect of the invention provides a system for monitoring the location of a tube, the system comprising an antenna located substantially at an outlet of the tube; a wireless transmitter coupled to the antenna for the transmission of wireless signals; a sensor located substantially at said outlet, the sensor being arranged to generate an output which indicates at least one sensed characteristic of an environment in which the outlet of the tube is located in use, and wherein said sensor is coupled to said wireless transmitter to cause said transmitter to transmit via said antenna, during use, a wireless signal which is indicative of said sensor output; and a remote receiver device for receiving said wireless signal, the receiver device including means for determining the strength of said received wireless signal, and means for determining said sensor output from said received wireless signal.

The system advantageously also includes means for indicating when the received signal strength is at a maximum or strongest level. Said indicating means is preferably included in said receiving unit.

In preferred embodiments, the system further includes a pH sensor located at, or adjacent, said end of the tube. The pH sensor and the antenna are preferably co-located, or substantially co-located. The pH sensor is arranged to measure the pH level at or adjacent the feeding end of the tube and to generate a corresponding electrical signal which is supplied to the transmitter, the transmitter causing the signal transmitted by the antenna to be adjusted or modulated in accordance with the electrical signal received from the pH sensor. For example, the frequency of the transmitted signal may be adjusted in accordance with the signal generated by the pH sensor. The receiving unit includes means for extracting the measured and transmitted pH level from signals it receives from the antenna.

Preferably, said transmitter is provided on or in said tube, more preferably at or adjacent said end.

Preferably, said pH sensor and/or said antenna and/or said transmitter are at least partially embedded in said tube. To this end, said end of the tube may include a solid, i.e. non-hollow, portion.

A second aspect of the invention provides a method for monitoring the location of a tube, especially an enteral feeding tube, in a system comprising an antenna located at, or adjacent, an end, especially a feeding end, of the tube; a transmitter coupled to the antenna for causing the antenna to emit a transmitted signal during use; and a receiver unit for receiving said transmitted signal, the receiver unit including means for determining the strength of said transmitted signal, the method comprising: with the end of the tube located inside a patient and the receiver unit located outside of the patient, positioning the receiver unit relative to the patient at a location where the determined strength of the transmitted signal is strongest; and determining that the end of the tube is located substantially in register with the positioned receiver unit. In preferred embodiments, the received unit, or at least a part of it, is adapted to be hand held or portable so that it may readily be passed over a patient's body.

In embodiments where the system includes said pH sensor, the method further includes determining the measured pH level from said transmitted signal; and determining, if said measured pH level is within a predetermined range and if said positioned receiver is located substantially in register with the patient's stomach, that the tube is correctly located.

To improve the accuracy of tube location detection, preferred embodiments of the invention combine pH sensing with wireless location technology, the latter being used to locate the position of the end or tip of the tube and the level of pH at that position.

The preferred wireless location technique requires that a micro transmitter antenna and a pH sensor are embedded into the fabric of a nasogastric, or similar, tube while a receiver, external to the patient's body, continually or intermittently receives a signal from the antenna. The pH sensor relays, using the wireless link, the level of pH at the tube tip to the external receiver and in doing so the position of the tip can be determined.

In preferred embodiments, the frequency of the wireless transmission can be adjusted or calibrated to reflect the pH level at the tube tip and hence the transmitted signal may also relay, using the wireless link, the level of pH at the tip.

Preferred embodiments enable simultaneous location of the tube tip and measurement of the pH level at the tip. This combined measurement significantly reduces the risk of feeding tube misplacement. This feature coupled with the simplicity and portability of the proposed wireless based system enables non-professional carers to actively participate in this aspect of care thereby reducing the intensive workload and cost placed on professional care providers.

Further advantageous aspects of the invention will become apparent to those ordinarily skilled in the art upon review of the following description of a specific embodiment and with reference to the accompanying drawings.

### BRIEF DESCRIPTION OF THE DRAWINGS

An embodiment of the invention is now described by way of example and with reference to the accompanying drawings in which:

FIG. 1 is a schematic view of a tube monitoring system embodying the invention;

FIG. 2 is a schematic view of the system in situ; and

FIG. 3 is a schematic view of the end of a tube forming part of said tube monitoring system, said tube end incorporating a pH sensor and an antenna.

### DETAILED DESCRIPTION OF THE DRAWINGS

Referring now to the drawings, there is shown, generally indicated as 10, a tube monitoring system embodying the invention. In the present embodiment, the system 10 is adapted for monitoring an enteral feeding tube and so the invention is described in this context. It will be understood that the invention is not limited to use with enteral feeding tubes and may instead, for example, be used to monitor an endotracheal tube.

The system 10 comprises a tube 12 (only partially shown), which in the present example comprises an enteral feeding tube, having a tip or end 14 which includes an outlet and is located, in use, in, or in close proximity with, a patient's stomach 16 (and which therefore may be referred to as the feeding end of the tube). The tube 12 has a body 18 which is at least partially hollow to define a bore 20. The outlet comprises one or more apertures 22 are formed in the body 18, typically at or adjacent the feeding end 14, exposing the bore 20 to the external environment. A mouth or inlet 13 is provided typically at the opposite end of the tube 12. Although not shown in the drawings, the tube 12 is passed into the stomach 16 via the nose (nasogastric), the mouth (orogastric) or surgical means such as Precutaneous Endoscopic Gastrostomy (PEG). During use, the tube 12 is positioned with the outlets 22 in or adjacent the stomach 16 so that food (typically liquid food) may be fed along the bore 20 and into the stomach 16 via the apertures 22. The, or each, aperture 22 is typically located at, or adjacent, the end 14 of the tube 12.

The tube 12 is provided with a sensor which in the preferred embodiment comprises a pH sensor 24. The pH sensor 24, which may for example comprise any suitable conventional pH sensor capable of generating an electrical signal corresponding to a detected pH level, is located at or adjacent the end 14 of the tube 12. In the preferred embodiment, the end 14 of the tube is solid, i.e. not hollow, and the pH sensor 24 is embedded, or at least partially embedded, in the solid end 14. Typically, a sensing part of the sensor 24 is exposed so that it may come into direct or intimate contact with the external environment, and in particular the contents of the patient's stomach, in order to measure the pH thereof. In alternative embodiments, alternative sensors may be used depending on the application. Typically, such sensors are of a type capable of sensing detecting or measuring one or more physical characteristics of the environment surrounding the tube (usually a liquid or gas).

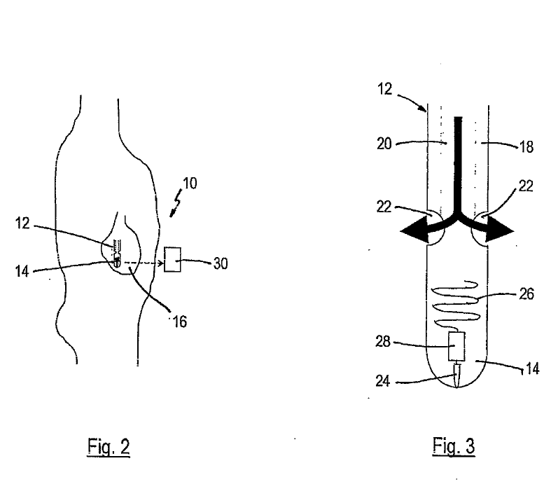

The tube 12 is also provided with an antenna 26. The antenna 26 is located at or adjacent the end 14 of the tube 12. In preferred embodiments, the antenna 26 is co-located, or substantially co-located, with the pH sensor 24. For example, the antenna 26 may co-located, or substantially co-located, with the pH sensor 24 in a direction that is axial of the tube 14. This may be achieved by locating the antenna 26 beside (in a transverse direction, i.e. perpendicular to the longitudinal axis of the tube 12) the pH sensor 24 or around the pH sensor 24. The latter option may be particularly convenient where the antenna 26 is a coil antenna. In the illustrated embodiment, the antenna 26 is located adjacent the pH sensor 24 in an axial direction. The antenna 26 shown in FIG. 2 is a coil antenna, although other conventional antenna types may alternatively be used. Advantageously, the antenna 26 is a micro antenna. In the preferred embodiment, the antenna 26 is embedded, or at least partially embedded, in the end 14 of the tube 12.

In the preferred arrangement, as illustrated, the outlet(s) 22 are located at the end of the bore 20 and the solid end 14 of the tube 12 is located beyond the outlet(s)22 with respect to the bore 20. As described above, the pH sensor 24 and antenna 26 are advantageously adjacent one another or substantially co-located. It is further preferred that the outlet(s) 22 are proximal or adjacent the sensor 24 and the antenna 26, conveniently adjacent the solid end 14.

The diameter of tube 12 may for example be approximately 2-8 mm and the length of the solid tip 14 may be approximately 5-10 mm. The tube 12 may be formed from any suitable material, typically plastics.

The system 10 further includes transmitter circuitry 28 for exciting the antenna 26 and causing it to transmit signals as is described in more detail hereinafter. The transmitter 28 is arranged to receive electrical signals from the pH sensor 24 indicating the detected level of pH and to drive the antenna 26 to transmit a corresponding signal, e.g. by modulating or adjusting the frequency of the signal transmitted by the antenna 26 in accordance with the signal provided by the sensor 24. The transmitter circuitry 28 may be of any suitable conventional design.

In the illustrated embodiment, the transmitter circuitry 28 is located at, and is preferably embedded in, the end 14 of the tube 12. An electrical power supply (not shown), conveniently in the form of one or more batteries, may also be provided at or in the end 14 for supplying power to the transmitter circuitry 28 and the pH sensor 24. Electrical power may be provided by any other suitable means, for example from an external power source which may supply the sensor 24 and circuitry 28 directly by wire (for example running along or embedded in the tube 12), or by means of inductive power coupling.

In an alternative embodiment, the transmitter circuitry 28 is provided separately from the tube 12 and is located, in use, externally of the patient's body. In such embodiments (not illustrated), the signal generated by the pH sensor 24 may be communicated to the external transmitter circuitry by a wired communication link, and the signal generated by the transmitter circuitry 28 may be communicated to the antenna 26 by the same, or a second, wired communication link. The or each wired communication link may conveniently be provided along, or be embedded in, the tube 12. Providing the transmitter circuitry 28 separately from the tube 12 may reduce the required size of the end 14 of the tube 12.

The system 10 also includes a receiver device, which in the illustrated embodiment takes the preferred form of a portable receiver unit 30. The receiver 30 may be provided separately from the tube 12 and includes receiving circuitry (including an antenna) for receiving signals transmitted by said antenna 26. The circuitry of the receiver 30 is preferably also arranged or designed to include means for determining the strength of the received signal, and optionally means for displaying or indicating signal strength to a user; and means for extracting the measured pH level from the received signal, and advantageously means for displaying or indicating said pH level to a user. The system 10 also includes means for displaying or indicating (audio and/or visual) the determined signal strength to a user, and/or preferably means for indicating when maximum signal strength is detected. The displaying/indicating means may be provided on a single receiver unit 30 as shown, although alternatively some of the components of the receiver 30 may be provided a separate external unit which may still be said to comprise part of the receiver device. The aforementioned determining, extracting, displaying or indicating means may be implemented using any suitable conventional electronic circuitry and audio and/or visual devices (e.g. display screen, lamp, buzzer etc.). In preferred embodiments, the receiver 30 is, or includes, a portable or hand held unit that is readily capable of being passed over the patient's body. The receiver 30 may include or be connectable to any convenient electrical power source (not shown).

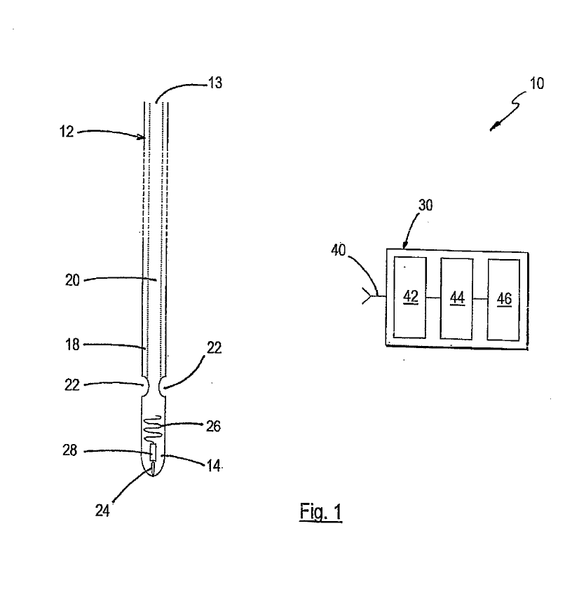

In the embodiment shown in FIG. 1, the receiver 30 includes an antenna 40, receiving circuitry 42 for receiving wireless signals via antenna 40 and processing circuitry 44 (which may be electronically implemented and/or may comprise computer software) for extracting pH measurements from received signals and for determining the strength of received signals. The receiver 30 further includes a display unit 46 which includes any suitable means for displaying the measured pH values and/or any suitable means for indicating the strength of the received signal and/or any suitable means for indicating when maximum signal strength is detected. As indicated previously, in alternative embodiments, any one or more of said display and/or indicating means, or other receiver components, may be provided on a separate unit (not shown) which may be in communication with the portable receiver unit 30 by any suitable means, e.g. a wireless or wired link.

In some embodiments, the receiver unit 30 includes means for generating a signal for energising the circuitry 24, 28 in the tip 14, the circuitry 24, 28 and antenna 26 being configured to be energised in this manner. This may be achieved by inductive coupling. Techniques similar to those used in passive RFID devices may be employed whereby a signal generated by the receiver 30 (or elsewhere) is received by the antenna 26 to provide sufficient power to energise the circuitry 24, 28 to perform at least one pH measurement and transmit a corresponding signal to the receiver 30. To this end, the receiver 30 may include a transmitter or transceiver.

During use, with the end of the tube 12 inserted into the patient, the pH sensor 24 monitors the pH level of the environment around the tip 14 of the tube 12 and provides a corresponding signal to the transmitter circuitry 28. The transmitter circuitry 28 causes the antenna 26 to transmit a signal modulated to carry information indicating the measured pH level. Conveniently, this is achieved by adjusting or modulating the frequency of the signal transmitted by the antenna 26 in accordance with the measured pH level, for example such that the frequency of the transmitted signal is proportional to the measured pH level. It is advantageous that the position of the tube 12 is continually monitored during use. It is preferred, therefore, that the antenna 26 is caused to transmit a signal continuously during use.

A user (not shown) holds the receiver 30 and passes it over the patient's body in the region where the tip or end of the tube 12 is expected to be. The receiver 30 measures the strength of the received signal. The receiver 30 (or other component of the system 10) displays or indicates the measured strength and/or indicates when the detected signal strength is at a maximum level. When maximum signal strength is indicated, the user may infer that the hand held receiver 30 is as close as possible to the antenna 26, and therefore to the end 14 of the tube 12. Hence, the user is able to determine, or at least gain a close approximation of, the position of the end 14 of the tube 12 by reference to the position of the hand held receiver 30 at maximum signal strength. The receiver 30 therefore locates the tube tip 14 by being positioned to receive the strongest signal from the antenna 26 and in this position it can be inferred that the tip 14 of the tube 12 is directly “behind” the receiver. To this end, it is advantageous that the antenna 26 is arranged to be, or to perform as, an isotropic or substantially isotropic antenna such that the generated. electromagnetic fields radiate substantially uniformly from the antenna 26.

In addition, the receiver 30 extracts from the received signal the measured pH level and this too gives an indication of the location of the end 14 of the tube 12 (typically, a measured pH level of between 3 and 8 indicates that the end 14 is in the stomach).

If the position of the tip 14 (as indicated by the position of the receiver 30), and the measured level of pH are consistent with the location of the stomach, then an inference may be made that the tube tip 14 is in the stomach cavity. It is considered that by detecting both the pH level and the position of the tube tip, the accuracy of tube location is significantly enhanced.

In an alternative embodiment, the receiver 30 may be adapted to transmit a signal to the transmitter circuitry 28 located at the tip 14 of the tube 12 in order to energise the transmitter circuitry 28 and so to excite or stimulate the antenna 26 as described above. This may be achieved by any suitable conventional means. Typically, the signal sent from the receiver 30 is received by the antenna 26 and used to energise or power the transmitter 28 for a limited period. During this period, the transmitter 28 drives the antenna 26 in the manner described above.

In some embodiments, two or more pH sensors may be provided and the detected pH level may be derived from a differential of their respective readings. This helps to reduce the effects that any drift in an individual sensor performance may have.

In an alternative embodiment, a receiver unit (similar to the receiver 30) may be embedded into a patient's clothing or fixed to the patient by some other means (e.g. a strap) and programmed to generate an alarm in the event that movements in the tube position and/or changes in the pH level at the tip of the tube are detected. It is also envisaged that the system may be adapted such that data related to the times of feed, the position of the tube and the level of pH at that time could be recorded remotely (e.g. in an electronic database or record for the patient) using a wireless link to the internet or other computer network.

The receiver 30 and the antenna 26 may communicate using any suitable form of wireless communication, conveniently a Radio Frequency (RF) link in which case the respective transmitting and receiving circuitry comprises RF circuitry.

The invention is not limited to the embodiments described herein which may be modified or varied without departing from the scope of the invention.

1. A system for monitoring the location of a tube, the system comprising an antenna located substantially at an outlet of the tube; a wireless transmitter coupled to the antenna for the transmission of wireless signals; a sensor located substantially at said outlet, the sensor being arranged to generate an output which indicates at least one sensed characteristic of an environment in which the outlet of the tube is located in use, and wherein said sensor is coupled to said wireless transmitter to cause said transmitter to transmit via said antenna, during use, a wireless signal which is indicative of said sensor output; and a remote receiver device for receiving said wireless signal, the receiver device including means for determining the strength of said received wireless signal, and means for determining said sensor output from said received wireless signal.

2. A system as claimed in claim 1, wherein said receiver device further includes means for indicating when a wireless signal received by said receiving device is at a maximum level.

3. A system as claimed in claim 1, wherein said sensor comprises a pH sensor arranged to measure the pH level of said environment.

4. A system as claimed in claim 1, wherein said sensor and said antenna are substantially co-located.

5. A system as claimed in claim 1, wherein the tube outlet, the antenna and the sensor are located substantially at a free end of the tube.

6. A system as claimed in claim 5, wherein at least one of said sensor and said antenna are embedded in a substantially solid portion of said tube.

7. A system as claimed in claim 6, wherein said solid tube portion is a tip portion at said free end of the tube.

8. A system as claimed in claim 5, wherein said transmitter is located substantially at said free end of the tube.

9. A system as claimed in claim 1, wherein said transmitter is provided separately from the tube and is connected to said antenna by means of at least one signal carrier.

10. A system as claimed in claim 9, wherein said at least one signal carrier is carried by said tube.

11. A system as claimed in claim 1, wherein said receiver device comprises a hand-held portable unit.

12. A system as claimed in claim 1, wherein said receiver device includes means for indicating to a user the sensor output obtained from the received wireless signal.

13. A system as claimed in any claim 1, wherein said receiver device includes means for indicating to a user the strength of the received wireless signal.

14. A system as claimed in claim 1, wherein said tube comprises an enteral feeding tube.

15. A method for monitoring the location of a tube in a system comprising an antenna located substantially at an outlet of the tube; a wireless transmitter coupled to the antenna for the transmission of wireless signals; a sensor located substantially at said outlet, the sensor being arranged to generate an output which indicates at least one sensed characteristic of an environment in which the outlet of the tube is located in use, and wherein said sensor is coupled to said wireless transmitter to cause said transmitter to transmit via said antenna, during use, a wireless signal which is indicative of said sensor output; and a remote receiver device for receiving said wireless signal, the receiver device including means for determining the strength of said received wireless signal, and means for determining said sensor output from said received wireless signal, said method comprising: determining said sensor output from the received wireless signal; determining the strength of the received wireless signal; and correlating said determined sensor output and signal strength to estimate the position of the tube.

16. A method as claimed in claim 15, wherein said receiver unit further includes a hand held portable receiver unit, the method further comprising: with the end of the tube located inside a patient and the receiver unit located outside of the patient, positioning the receiver unit relative to the patient at a location where the determined strength of the transmitted signal is strongest; and determining that the end of the tube is located substantially in register with the positioned receiver unit.

17. A method as claimed in claim 16, further including determining that if said sensor output is within a predetermined range and if said positioned receiver is located substantially in register with the patient's stomach, the tube is correctly located.
